SEQUENCE LISTING (3 sequences)

SEQ ID NO: 1 - DNA, 23 bp, Artificial Sequence
  Synthetic Oligonucleotide
cctccctattcagcactttgtcc

SEQ ID NO: 2 - DNA, 23 bp, Artificial Sequence
  Synthetic Oligonucleotide
tcagaagtagtaagggcgtggag

SEQ ID NO: 3 - DNA, 26 bp, Artificial Sequence
  Synthetic Oligonucleotide
aaacaagggaatttccctggcctcct

(19) United States
(12) Patent Application Publication
(10) Pub. No.: US 2015/0361417 A1
(43) Pub. Date: Dec. 17, 2015

(54) ASSAY FOR DETECTION OF JC VIRUS DNA

(71) Applicant: BIOGEN MA INC., Cambridge, MA (US)

(72) Inventor: Soma Ray, Needham, MA (US)

(73) Assignee: Biogen MA Inc., Cambridge, MA (US)

(21) Appl. No.: 14/763,895

(22) PCT Filed: Jan. 30, 2014
(86) PCT No.: PCT/US2014/013733

§ 371 Date: Jul. 28, 2015

Related U.S. Application Data
(60) Provisional application No. 61/758,463, filed on Jan. 30, 2013.

(51) Int. Cl. C12N 15/10; C12Q 1/70

(52) U.S. Cl. CPC C12N 15/101; C12Q 1/701; C12N 2710/22051

(57) ABSTRACT

In one aspect, the disclosure provides methods for isolating nucleic acid from a Cerebrospinal Fluid (CSF) sample. In one aspect, the disclosure provides methods for determining the amount of JC virus DNA in a sample.

### RELATED APPLICATIONS

This application claims the benefit under 35 U.S.C. §119(e) of U.S. provisional application No. 61/758,463, filed Jan. 30, 2013, which is incorporated by reference herein in its entirety.

### FIELD OF THE INVENTION

The invention is in the field of detection of nucleic acids in biological samples.

### BACKGROUND OF THE INVENTION

JC virus (JCV) is a human polyomavirus known to cause a rare disorder of the central nervous system (CNS) called progressive multifocal leukoencephalopathy (PML). The detection of JCV in the cerebrospinal fluid (CSF) is confirmatory of PML, but is technically challenging. Improved assays for the detection and quantification of JCV in the CSF are needed therefore.

### SUMMARY OF THE INVENTION

Various aspects of the invention provide, inter alia, methods and kits for isolating nucleic acid such as, for example, JC virus (JCV) DNA from a cerebrospinal fluid sample. According to aspects of the invention, biological samples thought to be virus free (e.g., CSF samples that are identified as JCV-free using standard techniques) do actually contain virus (e.g., JCV) that can be detected using techniques described herein. Detecting the presence of JCV in a sample of cerebrospinal fluid can be challenging because, in some instances, the virus is present in small quantities, which can lead to false-negative findings. Described herein in some aspects are novel nucleic acid detection methods and kits that reduce false negative results, in part, by increasing the yield of nucleic acid that can be isolated from a sample of cerebrospinal fluid. This can be achieved, in some instances, by providing more starting material than is used in current techniques (e.g., a larger volume of cerebrospinal fluid) and/or less carrier (e.g., lower concentration of RNA), though the invention is not limited in this regard.

Thus, in some aspects, the invention provides methods of isolating nucleic acid from a cerebrospinal fluid sample. The methods may comprise adding carrier nucleic acid and/or protease to a CSF sample, incubating the sample comprising the carrier nucleic acid and/or the protease, applying the incubated sample to a nucleic acid binding column, washing the column to which the sample was applied, and applying eluent to the column resulting in the isolation of the nucleic acid. In some embodiments, the volume of the CSF sample is at least 1 ml. In some embodiments, the carrier nucleic acid is carrier RNA. In some embodiments, the concentration of the carrier RNA in the cerebrospinal fluid sample is about 2.8 μg/ml or less (or is 2.8 μg/ml, or less). It should be understood that the invention contemplates methods that comprise (or consist of, or consist essentially of) any one or more of the foregoing steps, for example, any single step or the combination of any two, three, four, or five of the foregoing steps. The methods may also include additional steps, in some embodiments. The invention also contemplates performing a step(s) more than once, for example, it may be advantageous to perform the washing step two or more times. As another example, it may also be advantageous to perform the elution step more than once. In such instances, the eluted nucleic acid may be further concentrated by any standard method, for example, ethanol precipitation. The invention also contemplates omitting or substituting one or more of the foregoing steps. For example, in some instances, other solid phase extraction material (e.g., silica or other) may be used in place of a binding column to capture and/or purify the nucleic acid

In one aspect, the disclosure provides methods, kits and nucleic acids for determining the amount of JC virus (JCV) in a sample. JCV is a human polyomavirus that is known to cause a rare disorder of the CNS called progressive multifocal leukoencephalopathy (PML). JCV shares approximately 75% nucleotide homology with BK virus, another member of the polyomavirus family that commonly infects humans but does not cause PML.

Although initially identified as a major complication of HIV infection, in recent years, immunosuppressive therapeutic antibodies have been associated with an increased incidence rate of PML. In some embodiments, the detection of JCV in the CNS is an important step in confirming the presence of PML in a subject. Early detection of the JCV in CSF can be used as a basis for initiating early treatment for PML (e.g., before the progression of severe disease symptoms). Accordingly, early detection of JCV can be important for a good patient prognosis. In some embodiments, aspects of the invention relate to assay techniques and reagents that can increase the sensitivity of JCV detection in biological samples (e.g., CSF samples). In some embodiments, a real-time PCR assay described herein specifically detects JCV in human CSF with a sensitivity of 10 copies/mL.

Aspects of the invention relate to methods and compositions for confirming a diagnosis of PML in a subject who has signs or symptoms (e.g., early signs or symptoms) of PML. In some embodiments, the presence of JCV in the CSF of a patient is diagnostic of PML (for example, if the patient has one or more other signs or symptoms of PML). In some embodiments, the presence of JCV in the CSF of a subject can be useful to determine that the subject is at risk for PML. In particular, the invention provides methods and compositions for determining whether a subject is at risk of developing PML if the subject's immune system is compromised or suppressed. For example, aspects of the invention relate to determining whether a subject is suitable for an initial or continued treatment with an immunosuppressive agent (e.g., natalizumab or other immunosuppressive agent) by determining the subject's risk threshold for developing PML due to the presence of a JCV infection. It should be appreciated that when the presence of JCV in the CSF of a patient is used for a diagnosis of PML (e.g., an early diagnosis of PML), then the patient may be treated for PML and/or an immunosuppressive treatment that the patient is receiving may be discontinued if appropriate.

Accordingly, in some embodiments, aspects of the invention relate to a method for isolating nucleic acid from a Cerebrospinal Fluid (CSF) sample by adding carrier nucleic acid and protease to a CSF sample, incubating the sample comprising the carrier nucleic acid and the protease, applying the incubated sample to a nucleic acid binding column, washing the column to which the sample was applied, and applying eluent to the column resulting in the isolation of the nucleic acid.

In some embodiments, the volume of the CSF sample is at least 1 ml. In some embodiments, the carrier nucleic acid is carrier RNA. In some embodiments, the resulting concentration of the carrier RNA in the CSF sample is 2.8 microgram/ml or less. In some embodiments, incubating the sample comprises a first step of incubating the sample at room temperature (RT) and a second step of incubating the sample at a temperature that is above RT. In some embodiments, the incubating steps are 15 minutes long. In some embodiments, the temperature above RT is 56° C. In some embodiments, washing the column comprises adding a washing buffer to the column and spinning the column at a centrifugal force of 4000 g. In some embodiments, applying eluent comprises applying the eluent to the column for at least two times. In some embodiments, the eluent is incubated on the column for 5 minutes. In some embodiments, 30 microliters of eluent is applied. In some embodiments, the nucleic acid in the CSF sample is DNA, for example viral DNA (e.g., JCV DNA or other viral DNA).

In some embodiments, nucleic acid (for example DNA, e.g., viral DNA) is assayed for by performing a real-time polymerase chain reaction (Real-time PCR) to determine the amount of JC virus DNA. However, other detection methods (e.g., other PCR methods, other amplification methods, other hybridization based methods, one or more sequencing methods, etc.) may be used. In some embodiments, real-time PCR primers and probe are directed to the JC virus T antigen encoding sequence. In some embodiments, the sequences of the real-time PCR primers and probe are SEQ ID NOs:1-2 and SEQ ID NO:3, respectively.

In some embodiments, aspects of the invention relate to a method for determining the amount of JC virus DNA in a sample by performing real-time PCR on the sample, wherein the real-time PCR primers and probe are directed to the JC virus T antigen encoding sequences. In some embodiments, the sequences of the real-time PCR primers and probe are SEQ ID NOs:1-2 and SEQ ID NO:3, respectively.

In some embodiments, aspects of the invention relate to a kit for isolating nucleic acid from a Cerebrospinal Fluid (CSF) sample. In some embodiments, the kit comprises a protease, carrier nucleic acid, a nucleic acid binding column and/or instructions for use. In some embodiments, the kit further comprises real-time PCR primers and probes directed to a JC virus T antigen encoding sequence. In some embodiments, the sequences of the real-time PCR primers and probe are SEQ ID NOs:1-2 and SEQ ID NO:3, respectively.

In some embodiments, aspects of the invention relate to a nucleic acid primer that specifically hybridizes to (e.g., under stringent hybridization conditions) a conserved viral sequence (for example a conserved JCV sequence, e.g., a T antigen encoding sequence). In some embodiments, the nucleic acid is or includes the sequence of SEQ ID NO:1, SEQ ID NO:2, or SEQ ID NO:3.

These and other aspects of the invention are described in more detail herein.

### DETAILED DESCRIPTION OF THE INVENTION

In some embodiments, aspects of the invention relate to detecting JCV in a patient sample in order to evaluate the risk of PML in the patient. Although primary infection with JCV often occurs asymptomatically during childhood (Padgett & Walker, 1973), JCV is typically disseminated throughout the body, probably through viraemia (Ikegaya et al., 2004). While infection by JCV is asymptomatic in most subjects, infection may result in serious conditions (like PML) and even death in some subjects. Subjects most susceptible to PML are subjects that are immuno-compromised (e.g., AIDS patients) or subjects undergoing treatment with immuno-suppressants, for instance after organ transplant or to treat an inflammation related condition such as multiple sclerosis (e.g., using natalizumab or other immunosuppressive drug).

It is thought that JCV persists mostly in the kidneys in the absence of PML, and that PML is associated with the presence of JCV in the brain. Accordingly, in some embodiments, aspects of the invention relate to detecting JCV in CSF. However, methods and compositions of the invention also may be useful to detect JCV in urine, blood, renal tissue, or other patient samples.

In one aspect, the disclosure provides methods for isolating nucleic acid from a Cerebrospinal Fluid (CSF) sample. In some embodiments, the method comprises adding carrier nucleic acid and protease to a CSF sample, incubating the sample comprising the carrier nucleic acid and the protease, applying the incubated sample to a nucleic acid binding column, washing the column to which the sample was applied, and applying eluent to the column resulting in the isolation of the nucleic acid.

Cerebrospinal fluid is a fluid that surrounds and protects the brain and the spinal cord. The fluid generally is clear liquid that contains proteins and white blood cells. In general, CSF is obtained from a subject through a lumbar puncture (spinal tap). A lumbar puncture is a procedure that is unpleasant to a subject and the number of lumbar punctures should be minimized. A variety of disorders that affect the brain and/or the central nervous system, including meningitis, tumors of the brain, and hemorrhaging of the brain, can be diagnosed by analyzing the CSF. Viral infections of the brain, such as infections by the JC virus, can be diagnosed by detecting the presence of, and/or quantifying the amount of, viral DNA in the CSF. Because the amount of viral DNA (or viral RNA) in the CSF can be low, it is important to have diagnostic techniques that can accurately detect even small amounts of the virus.

### Isolating Nucleic Acids

In one aspect, the disclosure provides methods for isolating nucleic acids from a CSF sample. In some embodiments, the nucleic acid is DNA. In some embodiments, DNA from a DNA virus (e.g., JCV) is isolated from the CSF. However, it should be appreciated that methods described herein can be used to isolate other nucleic acids (e.g., DNA or RNA from other viruses or from other microbial or patient sources). In some embodiments, the nucleic acid is human nucleic acid (i.e., found in the human genome). In some embodiments, the nucleic acid is viral nucleic acid. In some embodiments, the nucleic acid is viral DNA. In some embodiments, the nucleic acid is JC virus DNA. In some embodiments, the nucleic acid is added to a CSF sample (i.e., “spiked”) prior to applying the methods for isolating provided herein (for example for use as a reference).

In one aspect, the disclosure provides methods for isolating nucleic acids from a CSF sample that use one or more components from commercially available nucleic acid isolation kits, such as kits provided by Qiagen, Promega and Epicentre. In some embodiments, the methods provided herein use one or more components from the QIAamp MinElute Virus Spin Kit (Cat #57704, Qiagen). However, it should be appreciated that the methods disclosed herein can also be practiced with components from other commercially available nucleic acid kits.

In some embodiments, the volume of CSF sample from which the nucleic acid is isolated is 0.5 ml or more, 1 ml or more, 1.5 ml or more, 2 ml or more, 2.5 ml or more, 3 ml or more, 5 ml or more, or at least 10 ml or more. In some embodiments, the volume of the sample of CSF from which the nucleic acid is isolated is 1 ml. It should be appreciated that a sample size of 1 ml is higher than the sample size that is generally used for the isolation of nucleic acids from a biological sample (e.g., 200 microliters or less) and from CSF in particular. According to some aspects of the invention, it is important to use a CSF volume of 1 ml or more in order to achieve sufficient sensitivity (e.g., to detect at least 10 copies of a JCV nucleic acid). It has been appreciated that a smaller volume (less than 1 ml) is not sufficient to provide sufficient sensitivity and/or reproducibility to confidently determine whether or not a patient has a positive PML diagnosis.

In some embodiments, carrier nucleic acid is added to the CSF sample from which the nucleic acid is isolated. The addition of carrier nucleic acid provides bulk to the nucleic acid to be isolated, minimizing the chance that the nucleic acid to be isolated is lost during one of the steps of the methods provided herein. In some embodiments, the carrier nucleic acid is RNA. In general the nature of the carrier nucleic acid will depend on the nature of the nucleic acid to be isolated (and analyzed). Thus, if the nucleic acid to be isolated is DNA, the carrier nucleic acid may be RNA (and vice versa). Upon completion of the isolation protocol, the no longer needed carrier nucleic acid RNA can easily be removed, for instance by addition of an RNAse. However, the nucleic acid to be analyzed and the carrier nucleic acid may be of the same nature, e.g., both DNA. In such cases the carrier nucleic acid will generally have a different size than the nucleic acid to be isolated (and analyzed) allowing for an easy separation of the two nucleic acids if so required.

In some embodiments, the resulting concentration of the carrier nucleic acid (e.g., RNA) in the CSF sample is 5 microgram/ml or less, 4 microgram/ml or less, 3 microgram/ml or less, 2 microgram/ml or less, 1 microgram/ml or less, or 0.5 microgram/ml or less. In some embodiments, the resulting concentration of the carrier nucleic acid (e.g., RNA) in the CSF sample is 2.8 microgram/ml or less. The resulting concentration, as used herein, refers to the concentration of the carrier nucleic acid in the CSF sample. Thus, the carrier nucleic acid may be prepared at a higher concentration and be diluted into the CSF sample. It was surprisingly found herein that the concentration of carrier nucleic acid used in the methods of the disclosure, which is lower than the concentrations generally used, resulted in increased yield of nucleic acid isolated from the CSF sample.

In some embodiments, the methods further include the addition of a protease to the CSF sample. While a CSF sample may contain less protein than other biological samples (e.g., blood), removal of proteins and polypeptide through the action of a protease may increase the yield of nucleic acid isolated from the CSF samples. Proteases for removing proteins and polypeptides from biological samples generally are non-specific proteases such as proteinase K and subtilisin. It should be appreciated that the additional components may need to be added, or the composition of the sample may need to be modified, to allow for the enzymatic activity of the proteases. Thus, a buffer comprising specific amounts of salt (e.g., NaCl or Mg-salts), or pH buffers, may be added. In addition, the sample may need to be incubated at a specific temperature to allow for optimized enzymatic conditions. After the protease reaction has occurred the protease may be removed or inactivated. Inactivation may be achieved for instance by adding a protease inhibitor, and/or adding a protease cofactor inhibitor, and/or increasing the sample temperature and/or changing the buffer conditions (e.g., by adding ethanol).

In some embodiments, carrier nucleic acid and protease are added to the CSF sample. In some embodiments, the carrier nucleic acid is added prior to the addition of the protease. In some embodiments, the protease is added prior to addition of the carrier nucleic acid. In some embodiments, the protease is added together with the carrier nucleic acid. A protease buffer can be added together with, prior to, or after the protease and/or the carrier nucleic acid are added. In some embodiments, additional components, such as a lysis buffer, can be added to the CSF sample. These additional components include lysozyme and chaotropic agents (e.g., guanidium-HCl and urea). In some embodiments, the additional component is the “lysis buffer” in a commercially available nuclei acid isolation kit. Generally the “lysis buffer” in these kits, is the first buffer used. In some embodiments, the buffer “AL” from the QIAamp MinElute Virus Spin Kit is added to the CSF sample.

It was surprisingly found herein that incubating the CSF sample comprising the carrier nucleic acid and protease at room temperature followed by a second incubation step at a temperature that is above room temperature (e.g., 56° C.), resulted in an increased yield in nucleic acid isolated from CSF. Thus, in some embodiments, the methods disclosed herein comprise a step of incubating the CSF sample comprising the carrier nucleic acid and protease at room temperature followed by a second incubation step at a temperature that is above room temperature. In some embodiments, depending on the enzyme preparation that is used, the temperature that is above room temperature is 30° C. or higher, 40° C. or higher, 50° C. or higher, 60° C. or higher, 70° C. or higher, 80° C. or higher, 90° C. or higher, up to 100° C. In some embodiments, the temperature that is above room temperature is between 50° C. and 60° C. In some embodiments, e.g., as described in the examples, the temperature that is above room temperature is 56° C. In some embodiments, the temperature that is above room temperature corresponds to the temperature at which the protease has the greatest activity.

In some embodiments, depending on the enzyme preparation that is used, the incubations steps are at least 1 minute, at least 2 minutes, at least 5 minutes, at least 10 minutes, at least 15 minutes, at least 20 minutes, at least 25 minutes, at least 30 minutes, at least 40 minutes, at least 50 minutes, at least 60 minutes, or up to 120 minutes long. The incubation step at room temperature and the incubation step at the temperature that is above room temperature can have the same length of time or can have a different length of time. In some embodiments, e.g., as described in the examples, the incubation step at room temperature and the incubation step at the temperature that is above room temperature are both 15 minutes long.

In some embodiments, following the incubation of the CSF sample comprising the carrier nucleic acid and the protease, the sample is purified by solid phase extraction methods such as, for example, column-based nucleic acid purification. These methods typically rely on the fact that the nucleic acid may bind to a solid phase (silica or other) depending on the pH and the salt content of buffer used, which may be a Tris-EDTA (TE) buffer or phosphate buffer. Generally, a nucleic acid purification method that can be used with various aspects of the invention includes:

adding a sample (e.g., a cerebrospinal fluid sample as used herein) to a binding column (or “spin” column), and the nucleic acid binds due to the lower pH (relative to the silanol groups on the column) and salt concentration of the binding solution, which may contain, e.g., buffer, a denaturing agent (such as guanidine hydrochloride), Triton X-100®, isopropanol and a pH indicator;

washing the column with, e.g., 5 mM KPO₄ pH 8.0 or similar, 80% ethanol (EtOH));

and eluting the nucleic acid with buffer or water.

In some embodiments, following the incubation of the CSF sample comprising the carrier nucleic acid and the protease, the sample is applied to a nucleic acid binding column. Nucleic acid binding columns are known in the art and include silica based columns (See e.g., U.S. Pat. No. 5,234,809), and anion exchange columns. In some embodiments a chaotropic reagent and/or salt may be added to the CSF sample prior to applying the CSF sample to the column to generate conditions that are optimal for binding of nucleic acid in the CSF sample to the nucleic acid binding column (e.g., a silica based column). The nucleic acid binding column used herein is not limited to a specific configuration, and includes bead based columns, columns whereby the nucleic acid binding components are covalently attached to the column, columns that work by gravity and columns that work by vacuum operation. In some embodiments, the nucleic acid binding column is an Eppendorf-tube sized “mini-column” that can fit in a bench top centrifuge. Eppendorf-tube sized “mini-columns” are provided by a number of suppliers including Qiagen (See e.g., QIAamp MinElute Virus Spin Kit), Epicentre and Promega.

Following the application of the CSF sample to the nucleic acid binding column, for example, the column may be washed by one or more washing buffers (e.g., Tris-based buffers at around pH 7.0 or around pH 8.0) and/or ethanol aliquots. The conditions of the washing buffers should be such that the bond/interaction between the nucleic acid and the nucleic acid binding column is not broken, and the nucleic acid remains bound to nucleic acid binding column. In some embodiments, the column is washed with a buffer comprising at least 70% ethanol. In some embodiments, the column is washed with a “washing buffer” provided in a commercial nucleic acid isolation kit. In some embodiments, the column is washed with buffer AW2 of the QIAamp MinElute Virus Spin Kit. In some embodiments, the column is washed with a buffer (such as AW2) in a first wash and ethanol in a second wash.

In some embodiments, the nucleic acid binding columns are “mini-columns”. In some embodiments, the washes may be removed by spinning the columns (e.g., in a bench-top centrifuge). It was surprisingly found herein that spinning the columns with a relatively low centrifugal force resulted in increased yield in nucleic acid isolated from the CSF sample. In some embodiments, the mini columns are centrifuged at a force less than 7000 g, less than 6000 g, less than 5000 g, less than 4000 g, less than 3000 g, less than 2000 g, or less than 1000 g to remove the washes. In some embodiments, the columns are centrifuged at 4000 g. In some embodiments, following the removal of the washes at relatively low centrifugal force, the columns are subsequently centrifuged at high centrifugal force to dry the columns.

Following the application of the CSF sample to the column and the washing of the column, eluent is applied to the column to harvest the nucleic acid form the column. The eluent is a buffer that will take up the nucleic acid that was bound to the nucleic acid binding column. Eluents include, water and phosphate buffer. In some embodiments, the eluents are DNAse and/or RNAse free. In some embodiments, the eluents also comprise a DNAse and/or RNAse inhibitor, and/or a DNAse and/or RNAse cofactor inhibitor. In some embodiments, the eluent includes a microbial toxin, such as sodium azide, to prevent microbial growth in the eluent. In some embodiments, the eluent is the “elution buffer” from a commercial nucleic acid isolation kit. In some embodiments, the eluent is AVE buffer the QIAamp MinElute Virus Spin Kit.

The volume of eluent that is applied to the nucleic acid binding column is generally a compromise between a larger volume, facilitating the uptake of a larger percentage of the nucleic acid from the column but resulting in a lower concentration of the isolated nucleic acid, and a smaller volume, resulting in a higher concentration of the isolated nucleic acid but at the expense of not taking up all the nucleic acid that was bound to the column. In some embodiments, a eluent volume of 1 microliter or more, 5 microliters or more, 10 microliters or more, 20 microliters or more, 30 microliters or more, 40 microliters or more, 50 microliters or more, 60 microliters or more, 70 microliters or more, 80 microliters or more, 90 microliters or more, 100 microliters or more, 200 microliters or more, or 500 microliters or more is applied to the column. In some embodiments, 30 microliters of eluent is applied to the column.

In some embodiments, the eluent is allowed to incubate on the column for 1 minute or longer, 2 minutes or longer, 5 minutes or longer, 10 minutes or longer, 20 minutes or longer, 30 minutes or longer, or 60 minutes or longer. In some embodiments, the eluent is allowed to incubate on the column for 5 minutes.

In some embodiments, the same eluent is applied to the column multiple times. Thus, in some embodiments, an eluent is applied to a column, allowed to incubate and the eluent (now including the nucleic acid) is removed from the column (e.g., by centrifugation) and subsequently reapplied to the column, allowed to incubate for a second time, and removed for the second time. In some embodiments, the same eluent is applied to the column two times, three times, four times, up to five times or more. In some embodiments, the same eluent is applied to the column two times.

In some embodiments, the 30 microliters of eluent is applied to the column, allowed to incubate for 5 minutes, removed from the column (e.g., by centrifugation), reapplied to the column, allowed to incubate for another 5 minutes and removed from the column.

Once the eluent, now including nucleic acid isolated from the CSF sample, has been removed from the column it can be stored at an appropriate temperature (e.g., 4° C., −20° C.) and/or the nucleic acid in the eluent can be analyzed (e.g., the sequence and/or the amount determined).

### Nucleic Acid Amplification

In one aspect, the disclosure provides methods for determining the amount of nucleic acid in a sample. In some embodiments, the nucleic acid is DNA. In some embodiments, the nucleic acid is viral nucleic acid. In some embodiments, the nucleic acid is viral DNA. In some embodiments, the nucleic acid is JC virus DNA. In some embodiments, the nucleic acid is isolated from a CSF sample. In some embodiments, the nucleic acid is isolated from a CSF sample by any of the methods disclosed herein. In some embodiments, the nucleic acid is JC virus DNA isolated from a CSF sample. In some embodiments, the nucleic acid is JC virus DNA isolated from a CSF sample by a method of adding carrier nucleic acid and protease to the CSF sample, incubating the sample comprising the carrier nucleic acid and the protease, applying the incubated sample to a nucleic acid binding column, washing the column to which the sample was applied, and applying eluent to the column.

However, it should be appreciated that aspects of the invention (e.g., purification and/or amplification techniques) may be used in combination with any suitable technique and/or matrix for binding and/or isolating nucleic acid (e.g., from the CSF).

In one aspect, the disclosure provides methods for determining the amount of nucleic acid in a sample comprising performing a Real-Time Polymerase Chain Reaction (Real Time-PCR), also called real-time quantitative PCR on the sample. Methods of real-time PCR to determine the amount of viral nucleic acid in a sample are well established (See e.g., McKay et al., Real-time PCR in virology, Nucl. Acids Res. 2002, 20:1292). Briefly, in real-time PCR two primers and a nucleic acid probe that can hybridize to a sequence of interest (e.g. a viral DNA sequence) are added to a sample. If the sequence of interest is present that sequence will be amplified through binding of the PCR primer and a PCR reaction. The PCR nucleic acid product will be detected/quantified through binding by the probe. Generally, the nucleic acid probe includes a reporter element such as a fluorescent label (e.g., 6-carboxyfluoescein, acronym: FAM) and a quencher, (e.g., tetramethylrhodamine, acronym: TAMRA). Prior to the binding to the PCR reaction product the fluorescent label is quenched and no fluorescence is observed. If the sequence of interest is present, the probe will bind to PCR-generated copies of the sequence (note that the probe also may bind to the target sequence if the target is present). Binding of the probe will result in physical separation of the quencher from the fluorescent label resulting in a fluorescent signal. In some embodiments, the fluorescent tag is released by the 5′ nuclease activity of the polymerase (e.g., Taq polymerase). The strength of the signal will be proportional to the amount of sequence of interest present allowing for the determination of the amount (e.g., the copy number) of the sequence of interest present. Generally the amount is benchmarked to samples with known quantities of the sequence. A number of commercial entities provide materials, including “wet-lab” components such as the polymerase, kits, and the hardware to run the real-time PCR experiment. Suppliers include Qiagen, Invitrogen, Applied Biosystems and Bio-Rad.

In one aspect, the disclosure provides methods for determining the amount of JC virus DNA in a sample comprising performing a Real-Time Polymerase Chain Reaction. In some embodiments, the Real-time PCR primers and probes are directed to the JV virus T antigen. In some embodiments, the primers correspond to the nucleic acid sequences 5′ CCT CCC TAT TCA GCA CTT TGT CC 3′ (SEQ ID NO:1) and 5′ TCA GAA GTA GTA AGG GCG TGG AG 3′ (SEQ ID NO:2), and the probe sequence corresponds to 5′-AAA CAA GGG AAT TTC CCT GGC CTC CT- 3′ (SEQ ID NO:3). In some embodiments, the probe fluorescent label is FAM and quencher is TAMRA. In some embodiments, the fluorescent label is on the 5′ end of the probe and the quencher is on the 3′ end. In some embodiments, the probe is 5′ FAM-AAA CAA GGG AAT TTC CCT GGC CTC CT-TAMRA 3′ (SEQ ID NO:3). However, it should be appreciated that alternative fluorescent labels, quenchers and/or alternative positioning of the fluorescent label and/or quencher on the probe sequence are also encompassed by the disclosure.

While the JV virus T antigen sequence had been used as a target sequence for real-time PCR previously (See Ryschkewitsch et al., J of Virological methods 2004, 121: 217), it was found herein that the combination of primers with SEQ ID NOs 1 and 2 and a probe of SEQ ID NO:3 provided superior results. However, in some embodiments, one or more other probe or primers (e.g., that are targeted to the JCV T antigen sequence) may be used.

It also should be appreciated that other amplification-based (e.g., PCR, etc.), hybridization-based, sequencing-based, and/or other detection techniques may be used (e.g., using one or more primers or probes described herein).

### Nucleic Acids

In one aspect, the disclosure provides isolated nucleic acids. In some embodiments, nucleic acids useful to detect JCV are specific for JCV (e.g., relative to BK virus or other virus nucleic acid that may be present in a biological sample). In some embodiments, the nucleic acids are complementary to JCV sequences but not to sequences from other viruses. In some embodiments, nucleic acids useful for detecting JCV are designed to detect conserved JCV regions (e.g., the nucleic acids are complementary, for example 100% complementary to, conserved JCV genomic regions) in order to detect the presence of JCV regardless of whether other variant sequences are present in the JCV genome. In some embodiments, the nucleic acids are primers and probes directed to (e.g., complementary to, for example 100% complementary to) either strand of the JC virus T antigen encoding sequences. In some embodiments, the nucleic acids allow for the determination of the amount of JC virus in a sample by real-time PCR. In some embodiments, the isolated nucleic acid comprises SEQ ID NO:1. In some embodiments, the isolated nucleic acid comprises SEQ ID NO:2. In some embodiments, the isolated nucleic acid comprises SEQ ID NO:3. In some embodiments, the isolated nucleic acid consists of SEQ ID NO:1. In some embodiments, the isolated nucleic acid consists of SEQ ID NO:2. In some embodiments, the isolated nucleic acid consists of SEQ ID NO:3.

In some embodiments, the isolated nucleic acid is a nucleic acid primer comprising SEQ ID NO:1. In some embodiments, the isolated nucleic acid is a nucleic acid primer comprising SEQ ID NO:2. In some embodiments, the isolated nucleic acid is a nucleic acid probe comprising SEQ ID NO:3. In some embodiments, the isolated nucleic acid is a nucleic acid primer that consists of SEQ ID NO:1. In some embodiments, the isolated nucleic acid is a nucleic acid primer that consists of SEQ ID NO:2. In some embodiments, the isolated nucleic acid is a nucleic acid probe that consists of SEQ ID NO:3. The isolated nucleic acids disclosed herein may further have one or more functionalities (e.g., a fluorescent label). In some embodiments, the nucleic acid corresponding to SEQ ID NO:3 is a nuclei acid probe that includes a fluorescent label and a quencher. In some embodiments, the nucleic acid probe corresponding to SEQ ID NO:3 is the probe 5′ FAM-AAA CAA GGG AAT TTC CCT GGC CTC CT-TAMRA 3′ (SEQ ID NO:3).

### Kits

In one aspect, the disclosure provides kits for the isolating nucleic acid from a Cerebrospinal Fluid (CSF) sample. In some embodiments, the kits comprise a protease, carrier nucleic acid, a nucleic acid binding column and instructions for use.

In some embodiments, the kits further comprise real time-PCR primers and probes directed to the JC virus T antigen. In some embodiments, the sequences of the Real-time PCR primers and probe are SEQ ID NOs:1-2 and SEQ ID NO:3, respectively.

In some embodiments, the present invention relates to a kit for isolating and or detecting the presence of JCV in a sample from a patient (e.g., from a human CSF sample). Accordingly, aspects of the invention relate to kits containing one or more components for isolating and preparing nucleic acids and/or one or more components for assaying for the presence and/or amount of a nucleic acid having a specified sequence. In some embodiments, a kit contains one or more buffers and/or other solutions for isolating JCV particles and/or JCV nucleic acid from a biological sample (e.g., a CSF sample), and optionally instructions for performing one or more isolation steps. In some embodiments, a kit contains one or more reagents for detecting a JCV nucleic acid in a sample. For example, a kit may include nucleic acid having a specified sequence. In some embodiments, the nucleic acid (e.g., a nucleic acid primer) may be provided as a dried powder (e.g., a lyophilized preparation). In some embodiments, the nucleic acid may be provided in solution. The solution may be diluent, a buffer, a salt solution, an aqueous solution, or other solution, including, for example, water. The solution may contain a known (e.g., predetermined) concentration of the nucleic acids. The kit may contain instructions for diluting the nucleic acid solution to one or more appropriate concentrations defined for one or more specified ingredients that are to be marked for subsequent authentication or quality control purposes. In some embodiments, a kit may contain one or more oligonucleotides (e.g., PCR primers) that can be used to detect the presence, in a biological sample (e.g., a CSF sample), of a nucleic acid having a specified sequence. A kit also may contain one or more enzymes and/or other reagents for performing a nucleic acid isolation, detection, and/or quantification assay of the invention. In some embodiments, a kit may contain a reference sequence and/or a reference nucleic acid having a specified sequence of interest. A reference level (e.g., information about a reference level) and/or a reference sample containing a nucleic acid at a reference level also may be provided in a kit. Such information and/or nucleic acids can be used as controls. In some embodiments, a kit also may include instructions for isolating nucleic acids (e.g., JCV nucleic acids) from a patient sample (e.g., a CSF sample).

In some embodiments, a kit comprises at least one container means having disposed therein one or more reagents (e.g., wash buffers, lysis buffers, proteases, elution buffers, etc.) and/or nucleic acids (e.g., PCR primers, detection probes, etc.) described herein. In certain embodiments, the kit further comprises other containers comprising one or more other reagents or probes. A kit also may contain detection reagents. In some embodiments, one or more probes in the kit may be labeled. In some embodiments, the kit may include reagents for labeling the probe (e.g., before or after contact with a JCV nucleic acid). Examples of detection reagents include, but are not limited to radiolabels, fluorescent labels, enzymatic labels (e.g., horse radish peroxidase, alkaline phosphatase), and affinity labels (e.g., biotin, avidin, or streptavidin).

In detail, a compartmentalized kit includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. In some embodiments, a kit may include a container which will accept the test sample, a container which contains the probe or primers used in the assay, containers which contain wash reagents (such as phosphate buffered saline, Tris-buffers, and the like), and containers which contain the reagents used to detect the hybridized probe, amplified product, or the like.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teaching that is referenced hereinabove.

### EXAMPLES

### Example 1

### DNA Extraction from Cerebrospinal Fluid (CSF)

### Materials and Methods

The QIAamp MinElute Virus Spin Kit (Cat #57704, QIAGEN) protocol was modified for processing human CSF samples. The following buffers were used for the DNA extraction
Buffer AW2 was prepared by adding 30 mL ethanol (96-100%) to the bottle containing 13 mL of Buffer AW2 concentrate and mixed thoroughly. The buffer was stored at ambient temperature.
A QIAGEN Protease was prepared by adding 1.4 mL buffer AVE to a bottle of lyophilized QIAGEN protease and mixed gently. The protease enzyme was stored at 2-8° C.
A carrier RNA Solution (1 μg/μL): was prepared by adding 310 μL buffer AVE to a tube of lyophilized carrier RNA to make a 1 μg/μL solution and mixed by pulse vortexing. The carrier RNA was be stored at −20±10° C. and did not undergo more than three freeze-thaws. The final concentration of carrier RNA in buffer AL was 5.6 μg/mL. For instance, for n samples [(1.1)×(5.6)×(n)]μL of carrier RNA Solution was added to [(1.1)×(n)]mL Buffer AL. The reagent was mixed by gentle inversions and used the day of preparation.

### DNA Extraction

Frozen CSF was thawed to room temperature and centrifuged for 5 minutes at 5000 g. Following centrifugation, 1000 μL of CSF was pipetted into a 15 mL centrifuge tube. QIAGEN Protease (125 μL) and AL buffer-carrier RNA solution (5.6 μg/mL, 1000 μL of) was added to the CSF.

The sample was vortexed for 15 seconds and incubated at room temperature for 15 minutes followed by incubation at 56° C. for 15 min in a water bath.

Following the incubations, 1250 μL of ethanol (96-100%) was added to the sample and mixed thoroughly by pulse-vortexing for 15 s. The lysate was subsequently incubated for 7 minutes at room temperature (15-25° C.).

The lysate was processed using the QIAvac 24 Plus vacuum manifold (Cat #19413, QIAGEN) by applying the whole lysate into a QIAamp Minelute column. If needed, multiple applications were used to apply the whole lysate. After binding, the column was washed with 500 μL of Buffer AW2 and centrifuged at 4000 g for 1 minute, followed by a wash with 500 μL of ethanol (96-100%) and centrifugation at 4000 g for 1 minute.

The QIAamp Minelute column was dried by centrifugation at 13000 g for 3 minutes followed by a centrifugation at 13000 g for 2.5 minutes with the cap of the column opened.

When the column was dry, it was placed in a clean DNase-free microcentrifuge tube and 30 μL of Buffer AVE was applied to the center of the membrane and incubated for 5 minutes. After incubation, the tube was centrifuged at full speed for 1 minute. In order to increase the amount of DNA eluted, the eluate was removed from the tube and reapplied to the center of the membrane followed by incubation for 5 minutes and centrifugation at full speed for 1 minute.

Following extraction, 1 μL of DNA was used for DNA quantitation and 20 μL is stored for PCR analysis.

### Example 2

### Real Time PCR Assay for Quantitation of JCV DNA

### Materials and Methods

Primers and probes were designed against the conserved region of the T-antigen gene of the JC virus genome and a BLAST search was performed to ensure the cross-reactivity. The sequence of the primers and probe is as follows:

| | Nucleotide Sequence |
|---|---|
| JCV Forward | 5′ CCT CCC TAT TCA GCA CTT |
| Primer | TGT CC 3′ |
| (SEQ ID NO: 1) | |
| JCV Reverse | 5′ TCA GAA GTA GTA AGG GCG |
| Primer | TGG AG 3′ |
| (SEQ ID NO: 2) | |
| JCV Probe | 5′ FAM-AAA CAA GGG AAT TTC |
| (SEQ ID NO: 3) | CCT GGC CTC CT-TAMRA 3′ |

Taqman real-time quantitative PCR was performed using the ABI 7900HT Sequence Detection System (Applied Biosystems). The real time PCR was run using the Taqman Universal PCR Master Mix (Applied Biosystems) and each reaction was prepared according to the following table:

TABLE 1

| | Catalog | Volume |
|---|---|---|
| | Number/ | in μL per |
| Master Mix Final | Manufacturer | reaction |
| 300 nM Forward | Custom | 0.15 |
| Primer (Stock = | Applied | |
| 100 uM) | Biosystems | |
| 300 nM Reverse | Custom | 0.15 |
| Primer (Stock = | Applied | |
| 100 uM) | Biosystems | |
| 200 nM Probe | Custom | 0.1 |
| (Stock = 100 uM) | Applied | |
| | Biosystems | |
| AmpliTaq Gold | Cat # N8080242 | 0.5 |
| DNA polymerase | Applied | |
| | Biosystems | |
| 10X IPC Exo Mix | Cat # 4308323 | 5 |
| 50X IPC DNA Mix | Applied | 1 |
| | Biosystems | |
| 1X Taqman Universal | Cat # 4304437 | 25 |
| PCR Master | Applied | |
| Mix (Stock = 2X) | Biosystems | |
| DNase/RNase | Cat # 10977-023 | 8.1 |
| free Water | Gibco (or similar) | |
| Total Volume | | 40 |

For each reaction, 40 μL of the above master mix was added to 10 μL of the DNA eluate on a MicroAmp® Optical 96-Well reaction plate (Cat # N8010560, Applied Biosystems) and subjected to PCR analysis according to the following steps:

1. 50° C. for 2 minutes—1 cycle
2. 95° C. for 10 minutes—1 cycle
3. 95° C. for 15 sec; 60° C. for 1 minute—50 cycles

A standard curve was prepared ranging from 10-10⁷ copies/mL using JC virus (Cat # VR-1583, ATCC) spiked into human CSF, that had been extracted using the optimized DNA extraction procedure and tested in duplicate. Each run also included a negative control consisting of unspiked CSF that underwent the same extraction process. The absolute copy number in a sample was quantitated by extrapolation from the standard curve using the ABI SDS software. All samples and standards were tested in duplicate and the average result from both the wells is reported as copies/mL.

Based on preliminary assay development, the limit of detection (LOD) was determined to be 10 copies/mL and the dynamic range is 10-10⁷ copies/mL. The specificity of the assay was evaluated against the closely related BK polyomavirus and no cross-reactivity was observed.

The reproducibility of the method of Example 1 is shown in the following table.

TABLE 2
Reproducibility of method of Example 1

| | Ct | Ct | Ct | Ct | Ct | | | |
|---|---|---|---|---|---|---|---|---|
| Human | Mean | Mean | Mean | Mean | Mean | Mean | | |
| CSF | Exp 1 | Exp 2 | Exp 3 | Exp 4 | Exp 5 | Ct | Std Dev | % CV |
| 10000 | 27.87 | 28.01 | 27.72 | 27.08 | 27.30 | 27.6 | 0.39 | 1.42 |
| 5000 | 28.74 | 29.74 | 29.11 | 28.14 | 28.94 | 28.94 | 0.58 | 2.01 |
| 1000 | 31.83 | 32.99 | 31.63 | 30.94 | 30.36 | 31.55 | 0.99 | 3.15 |
| 500 | 33.09 | 33.80 | 32.19 | 32.91 | 31.68 | 32.73 | 0.82 | 2.51 |
| 100 | 35.61 | 37.76 | 34.78 | 36.54 | 34.97 | 35.93 | 1.23 | 3.42 |
| 50 | 36.90 | 37.52 | 35.36 | 36.85 | 35.40 | 36.41 | 0.97 | 2.67 |
| 20 | 38.72 | N/A | 38.74 | 40.89 | 39.99 | 39.59 | 1.05 | 2.66 |
| 10 | 39.27 | 39.90 | 40.64 | 40.18 | 40.25 | 40.05 | 0.51 | 1.27 |
| 0 | ND | ND | ND | ND | ND | ND | N/A | N/A |

Ct: In a real time PCR assay a positive reaction is detected by accumulation of a fluorescent signal. The Ct (cycle threshold) is defined as the number of cycles required for the fluorescent signal to cross the threshold (ie exceeds background level). Ct levels are inversely proportional to the amount of target nucleic acid in the sample (ie the lower the Ct level the greater the amount of target nucleic acid in the sample).

The specificity of the method of Example 1 is shown in the following table.

TABLE 3
Specificity of JC virus detection of method of Example 1

| | | Copies/mL |
|---|---|---|
| | Copies/mL | (+ 5000 copies/mL |
| Viral DNA | (No JCV DNA) | JCV DNA) |
| HSV1 | 0 | 2555 |
| HSV2 | 0 | 2929 |
| CMV | 0 | 4785 |
| EBV | 0 | 4451 |
| VZV | 0 | 5333 |
| HHV7 | 0 | 6107 |
| HHV8 | 0 | 5273 |
| HHV6A | 0 | 3276 |
| HHV6B | 0 | 5109 |
| HTLV-1 | 0 | 1726 |
| HTLV-2 | 0 | 3856 |
| HIV1 | 0 | 10206 |
| HIV2 | 0 | 6265 |
| BKV | 0 | 7441 |
| JCV | 1210 | 6030 |

Specificity of primers/probe was assessed against 5000 copies/mL of different viral plasmid DNA ± 5000 copies/mL JCV DNA

### Example 3

### Comparison

The results of the method described under Example 1 were compared to the methods described in the “standard” protocol provided with the QIAamp MinElute Virus Spin Kit (Cat #57704, Qiagen). See for example pages 59-60 of the DNA Mini Kit handbook and pages 19-21 of the QIAamp MinElute Virus Spin Kit handbook. Various amounts of JC virus DNA copies were added to a CSF sample and DNA was isolated using both the “standard” protocol and the protocol described in Example 1. The copy number of the JC virus DNA in samples comprising the isolated DNA was determined using the RT-PCR protocol described under Example 2.

The “standard” extraction method resulted in an assay sensitivity of 500 copies/mL. The method described under Example 1 resulted in the detection of 10 copies/mL. (See Table below)

TABLE 4
Comparison method of Example 1 v. Standard protocol.

| | Mean Cₜ | Mean Cₜ |
|---|---|---|
| Copies/mL | (Example 1) | (Standard) |
| 10000000 | 20.66 | 23.80 |
| 1000000 | 23.66 | 27.05 |
| 500000 | 25.07 | 28.20 |
| 100000 | 27.64 | 30.06 |
| 10000 | 31.11 | 33.73 |
| 5000 | 32.60 | 35.15 |
| 1000 | 35.78 | 37.61 |
| 500 | 36.53 | 37.94 |
| 200 | 36.93 | Undetermined |
| 100 | 37.43 | 10.90 |
| 50 | 42.56 | Undetermined |
| 20 | Undetermined | Undetermined |
| 10 | 44.30 | Undetermined |
| 0 | Undetermined | Undetermined |

### EQUIVALENTS

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

The contents of all references, patents and published patent applications cited throughout this application are incorporated herein by reference in their entirety, particularly for the use or subject matter referenced herein.

What is claimed is:

1. A method for isolating nucleic acid from a Cerebrospinal Fluid (CSF) sample, the method comprising:
adding carrier nucleic acid and protease to a CSF sample,
incubating the sample comprising the carrier nucleic acid and the protease, applying the incubated sample to a nucleic acid binding column,
washing the column to which the sample was applied, and
applying eluent to the column resulting in the isolation of the nucleic acid.

2. The method of claim 1, wherein the volume of the CSF sample is at least 1 ml.

3. The method of claim 1, wherein the carrier nucleic acid is carrier RNA.

4. The method of claim 3, wherein the resulting concentration of the carrier RNA in the CSF sample is 2.8 microgram/ml or less.

5. The method of claim 1, wherein incubating the sample comprises a first step of incubating the sample at room temperature (RT) and a second step of incubating the sample at a temperature that is above RT.

6. The method of claim 5, wherein the incubating steps are 15 minutes long.

7. The method of claim 5, wherein the temperature above RT is 56° C.

8. The method of claim 1, wherein washing the column comprises adding a washing buffer to the column and spinning the column at a centrifugal force of 4000 g.

9. The method of claim 1, wherein applying eluent comprises applying the eluent to the column for at least two times.

10. The method of claim 1, wherein the eluent is incubated on the column for 5 minutes.

11. The method of claim 1, wherein 30 microliter of eluent is applied.

12. The method of claim 1, wherein the nucleic acid in the CSF sample is DNA.

13. The method of claim 12, wherein the DNA is viral DNA.

14. The method of claim 13, wherein the viral DNA is JC virus DNA.

15. The method of claim 14, further comprising performing a real-time polymerase chain reaction (Real-time PCR) to determine the amount of JC virus DNA.

16. The method of claim 15, wherein the Real-time PCR primers and probe are directed to the JC virus T antigen.

17. The method of claim 16, wherein the sequences of the Real-time PCR primers and probe are SEQ ID NO: 1, SEQ ID NO: 2 and SEQ ID NO:3, respectively.

18. A method for determining the amount of JC virus DNA in a sample, the method comprising:
performing Real-time PCR on the sample, wherein the Real-time PCR primers and probe are directed to the JC virus T antigen.

19. (canceled)

20. A kit for isolating nucleic acid from a Cerebrospinal Fluid (CSF) sample, the kit comprising a protease, carrier nucleic acid, a nucleic acid binding column and instructions for use.

21-22. (canceled)

23. A nucleic acid primer comprising SEQ ID NO:1, SEQ ID NO: 2 or SEQ ID NO: 3.

24-25. (canceled)
